(19) United States
(12) Patent Application Publication
(10) Pub. No.: US 2009/0018483 A1
(43) Pub. Date: Jan. 15, 2009

(54) Infrared Sample Chamber

(76) Inventors: Stephen D. Walker, Boulder, CO (US); Charles W. Henry, Boulder, CO (US); Peter E. Nelson, Boulder, CO (US); John E. Repine, Boulder, CO (US)

Correspondence Address: KOESTNER BERTANI LLP, 2192 Martin St., Suite 150, Irvine, CA, 92612, US

(21) Appl. No.: 11/777,961

(22) Filed: Jul. 13, 2007

(51) Int. Cl. A61M 37/00

(52) U.S. Cl. 604/6.08; 600/310

(57) ABSTRACT

A body fluid analysis apparatus comprises a unitary housing containing a single-celled chamber and having an entry portal for communicating body fluid between a patient body and the chamber. A barrier coupled at the entry portal prevents selected components of the body fluid from entering the chamber.

### BACKGROUND

The diabetic population is large and increasing. In 2005, 20.8 million Americans had diabetes, with over 1.5 million new cases diagnosed in the same year (American Diabetes Association (ADA) home page, www.diabetes.org). The diabetic population is growing by 7% annually, and shows little sign of abating (ADA home page, www.diabetes.org). Another 54 million Americans are pre-diabetic, meaning that they are already experiencing impaired glucose metabolism and up to 8% will become diabetic each year (Grady, D., Finding Whether Diabetes Lurks, New York Times, May 1, 2007).

Diabetic patients develop more medical complications and make up a disproportionate share of hospitalized patients. Diabetic or pre-diabetic patients comprise approximately 38% of all hospital admissions (Umpierrez G E, Isaacs S D, et al., Hyperglycemia: an independent marker of in-hospital mortality in patients with undiagnosed diabetes, Journal of Clinical Endocrinological Metabolism 2002; 87:978-982). Within hospital Intensive Care Units (ICUs) the percent of patients with impaired glucose metabolism is believed to be 56% (Davidson, Glucommander). Moreover, abnormal glucose metabolism also develops in seriously-ill non-diabetic individuals making the need for glucose assessment virtually universal.

Hospital care of patients with impaired glucose metabolism is shaped by three forces: (1) the vast number of diabetic patients; (2) the dramatic improvement in patient outcomes demonstrated by intensive insulin management; and (3) the very high cost of acquiring the frequent glucose measurements necessary to implement an intensive insulin therapy protocol.

Since the development of programs for intensive insulin management, improvement in the all-important measure of patient outcomes is well-documented. In 2001, Grete Van den Berghe, MD, published a seminal study that demonstrated the significant medical benefits derived by keeping an ICU patient's blood glucose levels between 80 and 110 mg/dl through highly managed insulin therapy (Van den Berghe G, et al., Intensive Insulin Therapy in Critically III Patients, New England Journal of Medicine (NEJM), Vol. 345, No. 19, Nov. 8, 2001). This study demonstrated very significant improvements in patient mortality, morbidity and length of hospitalization by aggressively using insulin to maintain low blood glucose levels and to decrease inflammation. Dr. Van den Berghe's initial findings have now been corroborated by many other studies in settings ranging from surgical ICUs (Furnary, A P, Zurr K J, et al, Continuous intravenous insulin infusion reduces the incidence of deep sternal wound infection in diabetic patients after cardiac surgical procedures. Annals of Thoracic Surgery 67:352-362, 1999) to general hospital wards (Newton, C A, Young, S, Financial implications of glycemic control, Endocrine Practice, Vol. 12, Jun. 8 2006, p. 43-48) to organ transplantations. So why doesn't every hospital use an intensive insulin management protocol? The answer is cost.

The current finger-stick approach for measuring glucose in ICU patients is too expensive and cumbersome. Intensive blood glucose monitoring necessitates dedicating one hospital technician per every twelve ICU beds to collect blood glucose samples from finger sticks. Even with the aggressive approach of intensive monitoring, a new glucose value is generated only once every hour per patient and that value provides only a single data point of information from which to adjust insulin delivery rates. No method exists for real-time assessment of the glucose level's direction or rate of change. In seriously ill individuals, glucose and insulin levels and other factors which affect these levels are changing very rapidly. Thus, a need exists for more frequent measurements and the valuable trend data that more measurements provide. Despite the savings and the improved outcomes, many medical and surgical ICU's have not been able to embrace the intensive insulin therapy approach because tight glycemic control is difficult to accomplish in terms of staffing, training, implementing and managing. In particular, ICU patients must be guarded carefully against the development of low blood sugars (hypoglycemia). However, this concern needs to be balanced against the desire to give as much insulin and to reduce blood sugars are much as possible. The reason that lower blood glucose levels and administering insulin is life-saving is unknown but may relate to an ability to reduce inflammation which is a common and contributing factor in the illness of these patients. Although no proof drives the concept, avoiding large swings in blood glucose levels is believed to be beneficial and can be best accomplished if more frequent glucose readings are made and insulin administration can be titered more specifically and frequently.

Assuming that the average cost for each hourly glucose reading is $10 and that the average length of stay in the ICU is 3 days (72 hours), then $720 is spent per patient visit to collect hourly glucose values.

### SUMMARY

An embodiment of a body fluid analysis apparatus comprises a unitary housing containing a single-celled chamber and having an entry portal for communicating body fluid between a patient body and the chamber. A barrier coupled at the entry portal prevents selected components of the body fluid from entering the chamber.

### BRIEF DESCRIPTION OF THE DRAWINGS

Embodiments of the invention relating to both structure and method of operation may best be understood by referring to the following description and accompanying drawings:

FIG. 1A is a schematic pictorial diagram showing a side view of an embodiment of a body fluid analysis apparatus that can be used to separate body fluids for optical analysis;

FIG. 1B is a schematic pictorial and block diagram illustrating a side view of another embodiment of a body fluid analysis apparatus;

FIGS. 2A through 2D are flow charts depicting one or more embodiments or aspects of a method for analyzing body fluid, for example for measuring a selected analyte;

FIG. 3 is a pictorial diagram showing a top view of an embodiment of a body fluid analysis apparatus that can be used to measure an analyte in body fluid for analysis; and

FIG. 4 is a pictorial diagram depicting another embodiment of a body fluid analysis apparatus for measuring an analyte in body fluid.

### DETAILED DESCRIPTION

An improved method for measuring blood glucose levels is of paramount importance today for life-saving effects in severely-ill, hospitalized patients. The new technology depicted herein has the potential to improve patient diagnosis and care, while also reducing the medical expenses of the many diabetic and non-diabetic ICU patients in hospitals worldwide.

Example hospital sectors in which the illustrative analyte concentration measurement device and methods can make an immediate impact include intensive care units (ICUs), surgical, and general hospital applications.

In the intensive care unit (ICU) estimates are that by 2008, 70% of the 53,805 ICU beds in the US will use an intensive insulin management protocol.

In surgical sectors approximately 10% of the 31 million surgical procedures performed annually in the US are potential users of the analyte concentration measurement device. Anesthesia procedures of two hours or longer create an acute need for critical information on glucose excursions.

In a general hospital sector approximately 38% of a hospital's patient population has diabetes, is pre-diabetic, or has nutritional monitoring requirements. Assuming that only 15% of the general hospital patient population is penetrated, the general hospital sector is still 1½ times larger than that of the ICU and surgical opportunities combined.

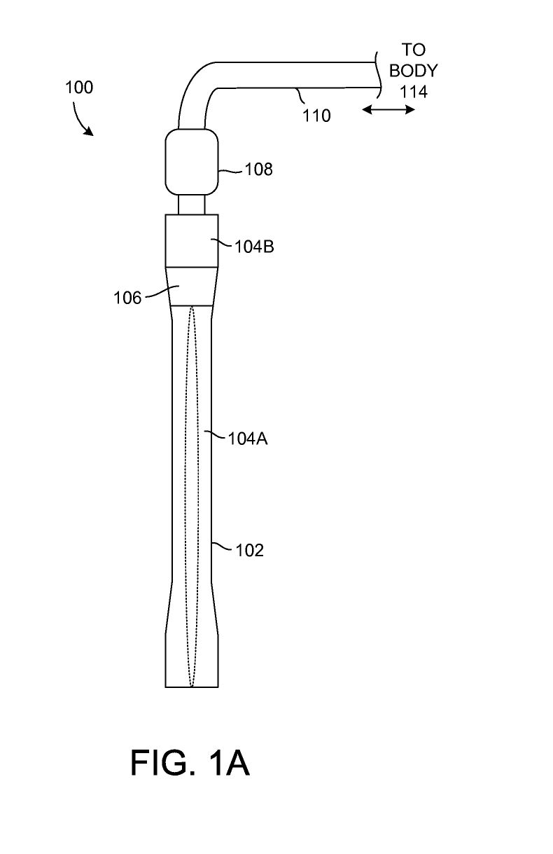

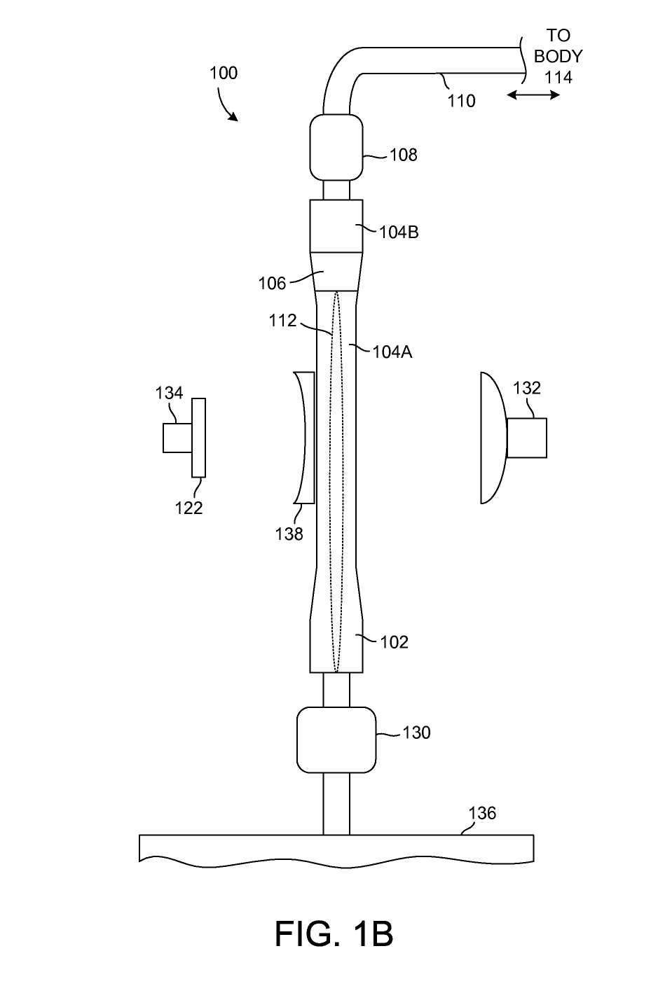

Referring to FIG. 1A, a schematic pictorial diagram illustrates a side view of an embodiment of a body fluid analysis apparatus 100 that can be used to separate body fluids for optical analysis. The illustrative body fluid analysis apparatus 100 comprises a single continuous sample chamber 102 containing a plurality of compartments 104A, 104B that hold body fluids for analysis, and at least one barrier 106 separating the compartments 104A, 104B that filters the body fluid into components with dissimilar compositions in different compartments 104A, 104B. The multiple compartments 104A, 104B comprising at least one optical compartment 104A. The sample chamber 102 is formed of a material in which the optical compartment 104A passes greater than 50% of 8-10 micrometer light.

In a particular embodiment, the single continuous two-compartment sample chamber 102 can be formed for holding a blood sample during infrared measurement of glucose concentration in the optical compartment 104A. Accordingly, the compartments 104A, 104B can include at least a compartment 104B for holding whole blood separated from the optical compartment 104A by a barrier 106 that prevents passage of red blood cells, for example in a specific embodiment, a barrier 106 with 1-2 micrometer pores that prevents passage of red blood cells.

In another example implementation, the compartments 104A, 104B can include at least a compartment 104B for holding whole blood separated from the optical compartment 104A by a membrane with 0.5-5.0 micrometer pores that prevents passage of red blood cells.

The body fluid analysis apparatus 100 can further comprise a body fluid interface 108 that couples the sample chamber 102 to a closed body fluid loop 110 of a patient body 114.

Removal of red blood cells (RBC) from blood is highly useful for accurate optical measurement of glucose. Beer's Law is given in equation (1) and shows glucose concentration in a liquid sample:

$${C}_{G} L {ɛ}_{λ} = - ln ( \frac{{I}_{1}}{{I}_{0}} ) , \qquad ( 1 )$$

where C_{G} is glucose concentration, L is the path length, ε_{λ} is the glucose absorption coefficient at wavelength λ, l₀ is the light intensity of wavelength λ at the detector when there is no sample in the optical path, and l₁ is the light intensity at the detector with a sample in the optical path. Equation (2) shows the composition of l₁ for a sample containing glucose:

I₁=I_{S}−I_{G}, (2)

where l_{S} is the intensity of the scattered light and l_{G} is the intensity of the light absorbed by glucose. Equation (2) demonstrates C_{G} is dependent on the intensity of scattered light and any change in l_{S} between two samples spaced temporally apart will be reflected as a change in C_{G}. Red blood cells (RBCs) have a large affect on l_{S} because of their complex shape. Changes in oxygenation, glucose, temperature and pH expand or contract the diameter of the RBCs and change the RBC index of refraction. Changes in RBC index of refraction affect how much scattered light reaches the detector. A higher RBC index of refraction spreads the scattered light out and lowers l_{S} Changes in l_{S} can be 2-3 times larger than l_{G} and obscure the glucose absorption. RBCs are typically removed by centrifuging the blood in a hospital's central laboratory. The method of obtaining RBC free samples is costly, time consuming, and eliminates the ability to measure real time glucose of critically ill patients at the bedside.

Referring to FIG. 1B, a schematic pictorial and block diagram illustrates a side view of another embodiment of a body fluid analysis apparatus 100 further comprising a vacuum pump 130 coupled to the sample chamber 102 which is formed to withdraw a body fluid sample 112 including plasma into the one or more optical compartments 104A through the barrier 106 that prevents passage of red blood cells (RBCs).

The body fluid analysis apparatus 100 can further comprise an emitter 132 and a photodetector 134 that is coupled across the optical compartment 104A of the sample chamber 102 from the emitter 132. The emitter 132 and photodetector 134 can be formed to pass infrared light through the optical compartment 104A onto the photodetector 134 to measure glucose concentration.

In an illustrative embodiment, the optical compartment 104A can be formed with an optical path length between the emitter 132 and photodetector 134 in a range of 10-50 micrometers (μm) to facilitate measurement of a selected analyte such as glucose. In some implementations, the optical compartment 104A can be formed with a sample volume in a range from 1-7 microliters. In a more specific implementation, the optical compartment 104A can be formed with a sample volume of approximately 3 microliters.

The sample chamber 102 can be molded from a material that is durable and has suitable optical properties. One suitable material is high density polyethylene (HDPE).

In some embodiments, the body fluid analysis apparatus 100 can further comprise an optical exit window 138 of the optical compartment 104A formed of a piano convex lens with a focal distance of 1-10 cm.

Some implementations of the body fluid analysis apparatus 100 can further comprise a saline pack 136 coupled to the sample chamber 102 that performs flushing of the compartments 104A, 104B after a measurement is acquired.

Referring to the system block and pictorial diagram shown in FIG. 1 B, a majority of patients in a hospital have some sort of catheter in a vessel. 80% of patients in the intensive care unit (ICU) have arterial catheters. The remainder has intravenous (IV) catheters for the administration of saline, insulin and other drugs. To obtain a bedside glucose sample, whole blood is extracted from the patient and drawn into the sample chamber 102 by the pump 130. An optical glucose measurement lasting about 30 seconds can be acquired when the sample fills the sample chamber 102. Pump flow is reversed after the glucose measurement and flushes the sample back into the patient's body with saline.

Whole blood enters the blood compartment 104B in the sample chamber 102. RBCs are prevented from entering the optical compartment 104A by a RBC barrier 106. Glucose is measured by directing 8-10 micrometer infrared (IR) light from an emitter 132 on one side of the optical compartment 104A, through the sample, through a lens 122 and onto a detector 134 on the other side. The sample path length through the optical compartment is 10-50 micrometers. The short sample path length is useful because water in the sample absorbs IR light. A further advantage of a short path length is that the volume of the sample is very small, 15 cubic micrometers. Specifications for the optical compartment material are most suitably non-blocking of infrared light, sufficient rigidity to hold 10-50 micrometer spacing, and non-dissolution when contacted by body fluid. Zinc selenide meets all specifications but is expensive and difficult to clean. A more desirable sample chamber material is low cost and disposable, for example high density polyethylene (HDPE) that has a transmission of 53% at 8.4 and 9.0 micrometers, and 64% at 9.7 micrometers.

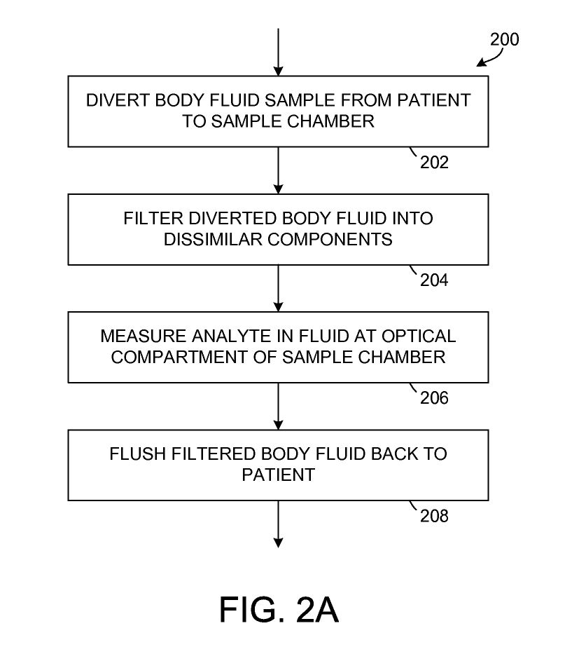

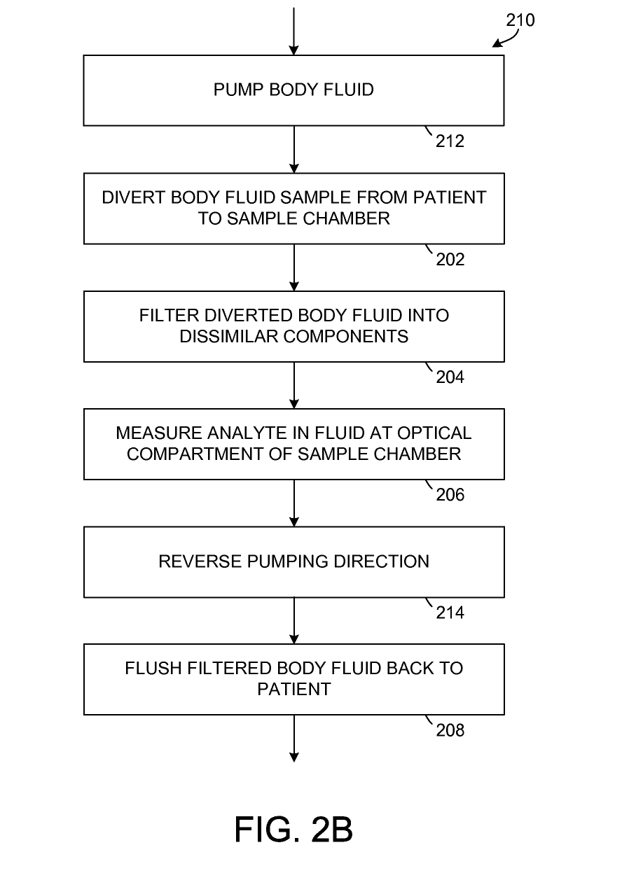

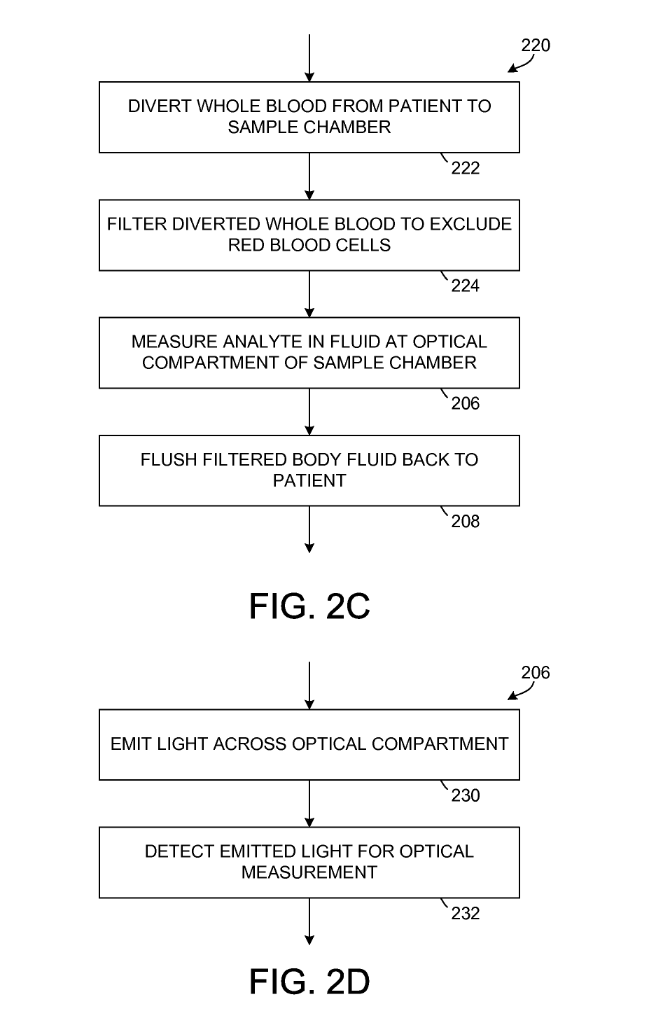

Referring to FIGS. 2A through 2D, flow charts illustrate one or more embodiments or aspects of a method for analyzing 200 body fluid, for example for measuring a selected analyte. The illustrative method 200 for analyzing body fluid comprises diverting 202 a body fluid sample from a patient body through a single continuous sample chamber containing multiple compartments and filtering 204 the diverted body fluid into components with dissimilar compositions in different compartments. The method 200 further comprises optically measuring 206 an analyte in the filtered body fluid in an optical compartment of the compartments and flushing 208 the filtered body fluid back to the patient body after optical measurement.

In a particular application, the filtering action 204 can comprise filtering red blood cells from the diverted body fluid wherein glucose concentration is optically measured 206 in the filtered body fluid in the optical compartment. The red blood cells can be filtered 204 from the diverted whole blood by passing the blood through a barrier that prevents passage of red blood cells, for example a barrier with 1-2 micrometer pores. In another implementation, filtering can be performed by passing the whole blood through a membrane with 0.5-5.0 micrometer pores.

Measurement accuracy can be improved by optically measuring 206 the analyte in the filtered body fluid in the optical compartment formed with an optical path length of 10-50 micrometers. Accuracy can further be improved through usage of the optical compartment formed with a sample volume in a range from 1-7 microliters, for example approximately 3 microliters.

In a particular example, the analyte in the filtered body fluid can be optically measured 206 in the optical compartment with an optical exit window formed of a piano convex lens with a focal distance of 1-10 cm.

The optical measurement and structural aspects of the measurement, specifically maintaining structural integrity during fluid movement, are facilitated by passing the body fluid sample through the single continuous sample chamber molded from high density polyethylene (HDPE).

Filtered body fluid can be flushed 208 back to the patient body after optical measurement by forcing saline into the sample chamber.

Referring to FIG. 2B, in some implementations the method 210 can further comprise pumping 212 body fluid so that the body fluid sample is diverted through the single continuous sample chamber. The pumping direction can be reversed 214 so that the filtered body fluid is flushed back to the patient body.

Referring to FIG. 2C, some method embodiments 220 can further comprise diverting 222 whole blood from the patient body through the single continuous sample chamber containing the multiple compartments and filtering 224 the diverted body fluid into fluid to exclude red blood cells in the optical compartment.

Referring to FIG. 2D, optically measuring 206 an analyte in the filtered body fluid can comprise emitting 230 light across the optical compartment of the sample chamber formed of a material so that the optical compartment passes greater than 50% of 8-10 micrometer light, and detecting 232 the emitted light for optical measurement.

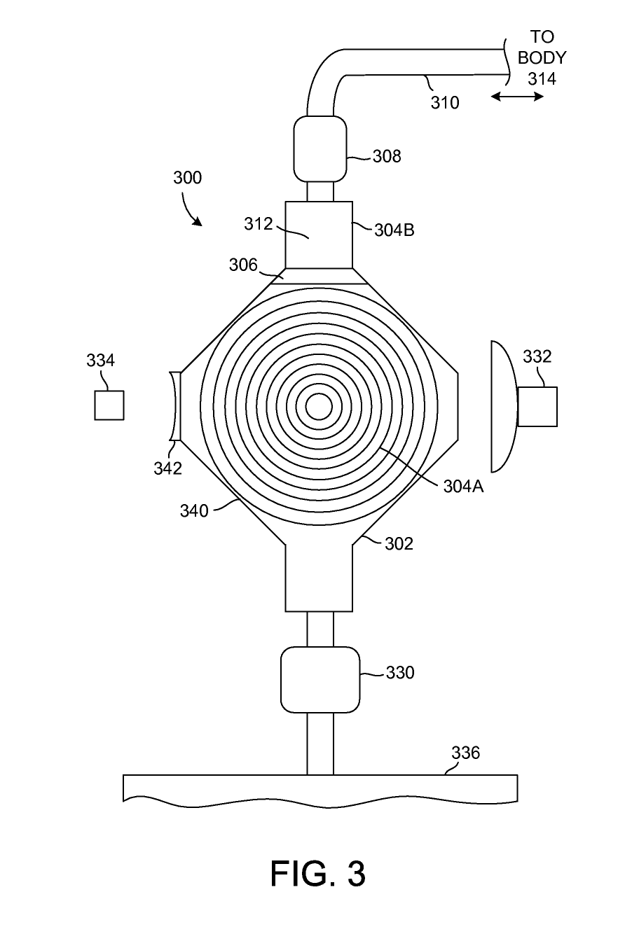

Referring to FIG. 3, a pictorial diagram depicts a top view of an embodiment of a body fluid analysis apparatus 300 that can be used to measure an analyte in body fluid for analysis. The illustrative body fluid analysis apparatus 300 comprises a unitary housing 340 containing a dual-compartment sample chamber 302 comprising a body fluid compartment 304B and an optical compartment 304A. The body fluid analysis apparatus 300 further comprises a body fluid interface 308 that couples the sample chamber 302 to a closed body fluid loop 310 of a patient body 314. A barrier 306 separates the body fluid compartment 304B from the optical compartment 304A and filters a body fluid component for optical analysis.

In an illustrative implementation, the housing 340 can be formed of a material such that the optical compartment 304A passes greater than 50% of 8-10 micrometer light to assist analyte measurement and analysis.

In a particular application, the housing 340 can contain a dual-compartment sample chamber 302 that holds a blood sample 312 during infrared measurement of glucose concentration in the optical compartment 304A.

The housing 340 is constructed from a material with suitable optical properties for analyte measurement. One example of a suitable material is molded high density polyethylene (HDPE).

The body fluid compartment 304B can be configured to hold whole blood that is separated from the optical compartment 304A by a barrier 306 that prevents passage of red blood cells, for example a barrier with 1-2 micrometer pores or a membrane with 0.5-5.0 micrometer pores in various implementations.

The optical compartment 304A of the housing 340 can further comprise an optical exit window 342 formed of a piano convex lens with a focal distance of 1-10 cm. Measurement and analysis of glucose concentration as the analyte can be aided by configuring the optical compartment 304A with an optical path length of 10-50 micrometers and with a sample volume in a range from 1-7 microliters, for example approximately 3 microliters.

The body fluid analysis apparatus 300 can further comprise a vacuum pump 330 coupled to the body fluid interface 308 that is formed to withdraw a body fluid sample comprising plasma into the optical compartment 304A through the barrier 306 that prevents passage of red blood cells (RBCs).

As shown in FIG. 3, the body fluid analysis apparatus 300 can further comprise an emitter 332 and a photodetector 334 coupled across the optical compartment 304A of the sample chamber 302 from the emitter 332. The emitter 332 and photodetector 334 can be formed to pass infrared light through the optical compartment 304A onto the photodetector 334 to measure glucose concentration.

In some embodiments the body fluid analysis apparatus 300 can comprise a saline pack 336 coupled to the housing 340 for flushing the optical compartment 304A and the body fluid compartment 304B after measurement.

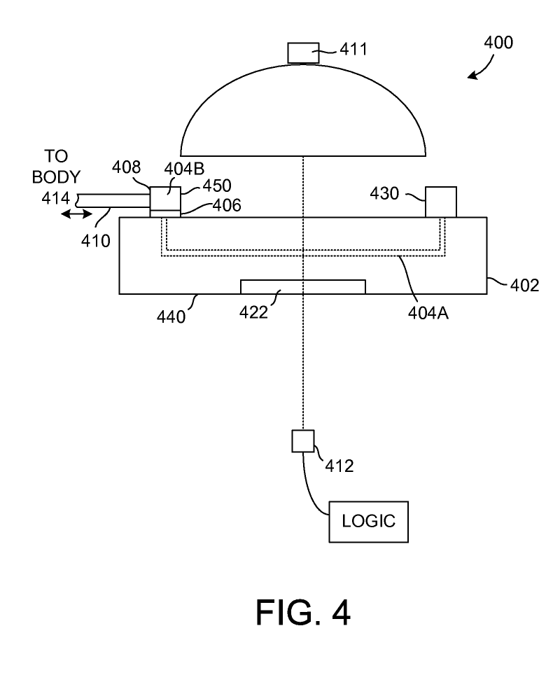

Referring to FIG. 4, a pictorial diagram depicts another embodiment of a body fluid analysis apparatus 400 for measuring an analyte in body fluid. The illustrative body fluid analysis apparatus 400 comprises a unitary housing 440 containing a single-celled chamber 402 and having an entry portal 450 for communicating body fluid between a patient body 414 and the chamber 402. The body fluid analysis apparatus 400 further comprises a barrier 406 coupled at the entry portal 450 that prevents selected components of the body fluid from entering the chamber 402.

The barrier 406 can be configured to divide the sample chamber 402 into a body fluid compartment 404B and an optical compartment 404A and functions to filter a body fluid component for optical analysis in the optical compartment 404A.

In a particular application, a two-compartment sample chamber 402 can hold a blood sample during infrared measurement of glucose. A first blood compartment 404A is separated from a second optical compartment 404B by a red blood cell (RBC) barrier 406 with 1-2 micrometer pores. Plasma is positioned in the first or optical compartment 404A with a vacuum pump 430 withdrawing a body fluid sample from the second or blood compartment 404B through the RBC barrier 406. Infrared light is emitted by an emitter 411 and passed through the optical compartment 404A onto a detector 412 to measure glucose concentration. The optical compartment 404A passes greater then 50% of 8-10 micrometer light. The optical path length is 10-50 micrometers. The optical compartment sample volume is 1-7 microliters. Both compartments are flushed with saline after the measurement is made.

The optical compartment 404A can have an exit window 442 in the form of a piano convex lens 422 with focal distance between 1-10 cm.

The housing 440 is constructed to hold a blood sample during infrared measurement of glucose concentration in the optical compartment 404A from a material that enables the optical compartment 404A to pass greater than 50% of 8-10 micrometer light. The sample chamber 402 can be molded out of high density polyethylene (HDPE).

The body fluid compartment 404B and optical compartment 404A are constructed with characteristics that enable improved measurement and analysis of a selected analyte. For example, measurement of glucose concentration is improved with the body fluid compartment 404A configured for holding whole blood separated from the optical compartment 404A by a barrier 406 with 1-2 micrometer pores or by a membrane with 0.5-5.0 micrometer pores thereby preventing passage of red blood cells.

In an illustrative implementation, the optical compartment 404A can have an optical path length of 10-50 micrometers and a sample volume in a range from 1-7 microliters.

The body fluid analysis apparatus 400 can further comprise a body fluid interface 408 that couples the sample chamber 402 to a closed body fluid loop 410 of a patient body 414.

The illustrative body fluid analysis devices and associated operating methods enable continuous, real-time blood glucose measurement at the bedside or by body-worn glucometers. The devices also prevent RBCs from entering the optical path, enabling accurate optical measurement of glucose because changes in scattered light caused by the change in RBC index of refraction no longer interfere with the glucose absorption.

The illustrative body fluid analysis devices also enable highly accurate analyte measurement with a very small sample size, for example less than 7 microliters of blood per measurement, an amount that is not significant compared to the patient blood volume. The depicted devices and associated methods can reinfuse 100% of the blood sample. No blood, RBCs or plasma are left over that require hazardous waste disposal. Furthermore, the body fluid analysis devices enable accurate analyte measurement without usage of reagents. A blood sample cannot be reinfused to the patient if reagents are used. Reagents also increase the cost of a glucose measurement.

The sample chamber can be constructed of low cost HDPE and does not require sterilization between patients.

Terms “substantially”, “essentially”, or “approximately”, that may be used herein, relate to an industry-accepted tolerance to the corresponding term. Such an industry-accepted tolerance ranges from less than one percent to twenty percent and corresponds to, but is not limited to, functionality, values, process variations, sizes, operating speeds, and the like. The term “coupled”, as may be used herein, includes direct coupling and indirect coupling via another component, element, circuit, or module where, for indirect coupling, the intervening component, element, circuit, or module does not modify the information of a signal but may adjust its current level, voltage level, and/or power level. Inferred coupling, for example where one element is coupled to another element by inference, includes direct and indirect coupling between two elements in the same manner as “coupled”.

The illustrative block diagrams and flow charts depict process steps or blocks that may represent modules, segments, or portions of code that include one or more executable instructions for implementing specific logical functions or steps in the process. Although the particular examples illustrate specific process steps or acts, many alternative implementations are possible and commonly made by simple design choice. Acts and steps may be executed in different order from the specific description herein, based on considerations of function, purpose, conformance to standard, legacy structure, and the like.

While the present disclosure describes various embodiments, these embodiments are to be understood as illustrative and do not limit the claim scope. Many variations, modifications, additions and improvements of the described embodiments are possible. For example, those having ordinary skill in the art will readily implement the steps necessary to provide the structures and methods disclosed herein, and will understand that the process parameters, materials, and dimensions are given by way of example only. The parameters, materials, and dimensions can be varied to achieve the desired structure as well as modifications, which are within the scope of the claims. Variations and modifications of the embodiments disclosed herein may also be made while remaining within the scope of the following claims.

What is claimed is:

1. A body fluid analysis apparatus comprising:
a single continuous sample chamber containing a plurality of compartments that hold body fluids for analysis;
at least one barrier separating the compartment plurality that filters the body fluid into components with dissimilar compositions in different compartments; and
the plurality of compartments comprising at least one optical compartment, the sample chamber formed of a material whereby the optical compartment passes greater than 50% of 8-10 micrometer light.

2. The apparatus according to claim 1 further comprising:
a body fluid interface that couples the sample chamber to a closed body fluid loop of a patient body.

3. The apparatus according to claim 1 further comprising:
a single continuous two-compartment sample chamber formed for holding a blood sample during infrared measurement of glucose concentration in the optical compartment.

4. The apparatus according to claim 1 further comprising:
the compartment plurality comprising at least a compartment for holding whole blood separated from the optical compartment by a barrier that prevents passage of red blood cells.

5. The apparatus according to claim 1 further comprising:
the compartment plurality comprising at least a compartment for holding whole blood separated from the optical compartment by a barrier with 1-2 micrometer pores that prevents passage of red blood cells.

6. The apparatus according to claim 1 further comprising:
the compartment plurality comprising at least a compartment for holding whole blood separated from the optical compartment by a membrane with 0.5-5.0 micrometer pores that prevents passage of red blood cells.

7. The apparatus according to claim 1 further comprising:
a vacuum pump coupled to the sample chamber and formed to withdraw a body fluid sample comprising plasma into the at least one optical compartment through a barrier that prevents passage of red blood cells (RBCs).

8. The apparatus according to claim 1 further comprising: an emitter; and
a photodetector coupled across the optical compartment of the sample chamber from the emitter, the emitter and photodetector formed to pass infrared light through the optical compartment onto the photodetector to measure glucose concentration.

9. The apparatus according to claim 1 further comprising:
the at least one optical compartment is formed with an optical path length of 10-50 micrometers.

10. The apparatus according to claim 1 further comprising:
the at least one optical compartment is formed with a sample volume in a range from 1-7 microliters.

11. The apparatus according to claim 1 further comprising:
the at least one optical compartment is formed with a sample volume of approximately 3 microliters.

12. The apparatus according to claim 1 further comprising:
a saline pack coupled to the sample chamber for flushing the compartment plurality after measurement.

13. The apparatus according to claim 1 further comprising:
an optical exit window of the at least one optical compartment formed of a piano convex lens with a focal distance of 1-10 cm.

14. The apparatus according to claim 1 further comprising:
the sample chamber molded from high density polyethylene (HDPE).

15. A method for analyzing body fluid comprising:
diverting a body fluid sample from a patient body through a single continuous sample chamber containing a plurality of compartments;
filtering the diverted body fluid into components with dissimilar compositions in different compartments;
optically measuring an analyte in the filtered body fluid in an optical compartment of the compartment plurality; and
flushing the filtered body fluid back to the patient body after optical measurement.

16. The method according to claim 15 further comprising:
pumping body fluid whereby the body fluid sample is diverted through the single continuous sample chamber; and
reversing pumping direction whereby the filtered body fluid is flushed back to the patient body.

17. The method according to claim 15 further comprising:
diverting whole blood from the patient body through the single continuous sample chamber containing the plurality of compartments; and
filtering the diverted body fluid into fluid excluding red blood cells in the optical compartment.

18. The method according to claim 15 further comprising:
emitting light across the optical compartment of the sample chamber formed of a material whereby the optical compartment passes greater than 50% of 8-10 micrometer light; and detecting the emitted light for optical measurement.

19. The method according to claim 15 further comprising:
filtering red blood cells from the diverted body fluid; and
optically measuring glucose concentration in the filtered body fluid in the optical compartment.

20. The method according to claim 15 further comprising:
filtering red blood cells from the diverted whole blood through a barrier that prevents passage of red blood cells.

21. The method according to claim 15 further comprising:
filtering red blood cells from the diverted whole blood through a barrier with 1-2 micrometer pores that prevents passage of red blood cells.

22. The method according to claim 15 further comprising:
filtering red blood cells from the diverted whole blood through a barrier with 1-2 micrometer pores that prevents passage of red blood cells.

23. The method according to claim 15 further comprising:
optically measuring the analyte in the filtered body fluid in the optical compartment formed with an optical path length of 10-50 micrometers.

24. The method according to claim 15 further comprising:
optically measuring the analyte in the filtered body fluid in the optical compartment formed with a sample volume in a range from 1-7 microliters.

25. The method according to claim 15 further comprising:
optically measuring the analyte in the filtered body fluid in the optical compartment formed with a sample volume of approximately 3 microliters.

26. The method according to claim 15 further comprising:
flushing saline into the sample chamber whereby filtered body fluid is forced back to the patient body after optical measurement.

27. The method according to claim 15 further comprising:
optically measuring the analyte in the filtered body fluid in the optical compartment with an optical exit window formed of a piano convex lens with a focal distance of 1-10 cm.

28. The method according to claim 15 further comprising:
diverting the body fluid sample through the single continuous sample chamber molded from high density polyethylene (HDPE).

29. A body fluid analysis apparatus comprising:
a unitary housing containing a dual-compartment sample chamber comprising a body fluid compartment and an optical compartment;
a body fluid interface that couples the sample chamber to a closed body fluid loop of a patient body; and
a barrier separating the body fluid compartment from the optical compartment and filtering a body fluid component for optical analysis.

30. The apparatus according to claim 29 further comprising:
the housing formed of a material whereby the optical compartment passes greater than 50% of 8-10 micrometer light.

31. The apparatus according to claim 29 further comprising:
the housing containing a dual-compartment sample chamber formed for holding a blood sample during infrared measurement of glucose concentration in the optical compartment.

32. The apparatus according to claim 29 further comprising:
the body fluid compartment configured for holding whole blood separated from the optical compartment by a barrier that prevents passage of red blood cells.

33. The apparatus according to claim 29 further comprising:
the body fluid compartment configured for holding whole blood separated from the optical compartment by a barrier with 1-2 micrometer pores that prevents passage of red blood cells.

34. The apparatus according to claim 29 further comprising:
the body fluid compartment configured for holding whole blood separated from the optical compartment by a membrane with 0.5-5.0 micrometer pores that prevents passage of red blood cells.

35. The apparatus according to claim 29 further comprising:
the optical compartment of the housing further comprising an optical exit window formed of a piano convex lens with a focal distance of 1-10 cm.

36. The apparatus according to claim 29 further comprising: the housing molded from high density polyethylene (HDPE).

37. The apparatus according to claim 29 further comprising:
a vacuum pump coupled to the body fluid interface and formed to withdraw a body fluid sample comprising plasma into the optical compartment through the barrier that prevents passage of red blood cells (RBCs).

38. The apparatus according to claim 29 further comprising: an emitter; and
a photodetector coupled across the optical compartment of the sample chamber from the emitter, the emitter and photodetector formed to pass infrared light through the optical compartment onto the photodetector to measure glucose concentration.

39. The apparatus according to claim 29 further comprising: the optical compartment has an optical path length of 10-50 micrometers.

40. The apparatus according to claim 29 further comprising:
the optical compartment has a sample volume in a range from 1-7 microliters.

41. The apparatus according to claim 29 further comprising:
the optical compartment has a sample volume of approximately 3 microliters.

42. The apparatus according to claim 29 further comprising:
a saline pack coupled to the housing for flushing the optical compartment and the body fluid compartment after measurement.

43. A body fluid analysis apparatus comprising:
a unitary housing containing a single-celled chamber and having an entry portal for communicating body fluid between a patient body and the chamber; and
a barrier coupled at the entry portal that prevents selected components of the body fluid from entering the chamber.

44. The apparatus according to claim 43 further comprising:
the barrier configured to divide the sample chamber into a body fluid compartment and an optical compartment and filtering a body fluid component for optical analysis in the optical compartment.

45. The apparatus according to claim 44 further comprising:
the housing formed for holding a blood sample during infrared measurement of glucose concentration in the optical compartment of a material whereby the optical compartment passes greater than 50% of 8-10 micrometer light.

46. The apparatus according to claim 44 further comprising:
the body fluid compartment configured for holding whole blood separated from the optical compartment by a barrier with 1-2 micrometer pores that prevents passage of red blood cells.

47. The apparatus according to claim 44 further comprising:
the body fluid compartment configured for holding whole blood separated from the optical compartment by a membrane with 0.5-5.0 micrometer pores that prevents passage of red blood cells.

48. The apparatus according to claim 44 further comprising:
the optical compartment has an optical path length of 10-50 micrometers and a sample volume in a range from 1-7 microliters.

49. The apparatus according to claim 43 further comprising:
a body fluid interface that couples the sample chamber to a closed body fluid loop of a patient body.
